(19) United States
(12) Patent Application Publication
(10) Pub. No.: US 2013/0289683 A1
(43) Pub. Date: Oct. 31, 2013

(54) DISTRIBUTED IMPLANT SYSTEMS

(71) Applicants: John Parker, Roseville (AU); Peter Single, Lane Cove (AU); Dean Karantonis, Maroubra (AU)

(72) Inventors: John Parker, Roseville (AU); Peter Single, Lane Cove (AU); Dean Karantonis, Maroubra (AU)

(73) Assignee: Saluda Medical Pty. Ltd., Eveleight ,NSW (AU)

(21) Appl. No.: 13/819,675

(22) PCT Filed: Aug. 31, 2011
(86) PCT No.: PCT/AU2011/001127

§ 371 Date: Jul. 18, 2013

(30) Foreign Application Priority Data
Aug. 31, 2010 (AU) 2010903899

(51) Int. Cl. A61N 1/05

(52) U.S. Cl. CPC A61N 1/0551
USPC 607/116; 29/825

(57) ABSTRACT

A distributed implantable neurostimulation system. One or more electrode arrays each have at least one electrode configured to be positioned at a desired implant location within the body. An implantable control unit is configured to selectively direct stimulus and/or telemetry instructions and power to each electrode of each array. A shared bus extends to each of the plurality of electrode arrays, the bus interconnecting each array with the implantable control unit. There is at least one electrode cell associated with each electrode array. The electrode cell obtains electrical power and command signals from the shared bus, and controls operation of each electrode associated with that electrode cell. The bus is connected to the control unit and/or the electrode cell by docking contacts of the bus to form electrical contact with contacts of the control unit and/or electrode cell.

### TECHNICAL FIELD

The present invention relates to implantable neuro-stimulating devices, and in particular the present invention provides components and a system for a distributed implant system.

### BACKGROUND OF THE INVENTION

Active implantable medical devices usually consist of an electronics module and an interface mechanism to tissue. Current implantable neuro-stimulators consist of a hermetically sealed electronics module which may contain one or more batteries, and which is interfaced to an electrode system. The connection from the electronics module to the electrode system may be either direct or through an implantable connector inserted into a connector block. The connector block, feed-through and batteries occupy a significant percentage of the volume of the device. The electronics module is typically hermetically sealed in a titanium or ceramic case, in part to protect the sensitive components inside the case from the corrosive environment of the body. The tissue interface for neuro-stimulator applications consists of a stimulating electrode that delivers an impulse to underlying nerve or tissue. The stimulating electrode can consist of a single contact, a strip of contacts, a two dimensional array of contacts or even more complex structures.

Currently such devices usually share very similar architectures and construction approaches. They have:
an electronics module to control the device's function. This module will contain information relating to the device's function either in the form of stored programmes and data and/or hardwired into the device's control circuitry;
a means of providing power (which can be via an inductive link and/or an implanted battery);
a means of communicating with an external programming and reporting device. Such an interface may be provided by an RF link, inductive coupling or other means;
an hermetic, biocompatible case to enclose the electronics and battery (where present);
a connector or feed-through structure to allow electrical signals and power to be passed from the inside of the case to the outside;
a lead assembly which connects the connector or feed-through assembly on the case to a tissue interface assembly; and
an interface to the underlying tissue for a functional or neuro-stimulator application.

There exist a range of limitations to these various system components. Common device architectures currently require that each tissue interface element (electrode) must have its own connecting element, such as an insulated wire, which must connect between the tissue interface and the control electronics within the hermetic case. This connecting element forms part of the lead assembly. When large numbers of electrodes are required there must be a similarly large number of connecting elements which can make the lead assembly bulky, stiff and possibly prone to failure.

Long leads are often required, and in some applications the resistance of the wire can lead to inefficient systems. The leads may travel significant distances under the skin from the site of implantation of the pulse generator to the target tissue for stimulation. In some cases the lead may be required to cross a joint (e.g. the neck as in the case of DBS for Parkinson's disease). The movement the lead experiences then may lead to fatigue failures and this requires special attention in design to prevent such failures from occurring. Also the inherent stiffness of some leads can cause them to migrate through soft tissue, causing biological problems.

All such devices must provide a way of connecting the conducting elements in the lead to the electronics inside the case. This connection is often made by a feed-through on the package. Connections between the feed-through and lead can be made in either a permanent way (e.g. the lead wires are welded to the feed-through contacts) called “hard wiring”, or in a re-connectable way by integrating a suitable connector socket assembly (e.g. an “IS connector”) into the case assembly and terminating the lead assembly in the mating “plug” assembly, called “connectorised”. Such high capacity feed-throughs and connectors are difficult to make. Current approaches limit channel counts for connectorised devices to perhaps as low as 16 channels, and limit hard-wired devices to around 25 channels.

While it is generally desirable to provide a connector arrangement in the manner described above, such connectors become more bulky as the number of channels increases. In many cases the size of the connector assembly can become a determining factor for the size of the implant as a whole. Even with modest channel counts, the use of a traditional multi-channel connector can mean that the electronics and battery case becomes too large to be optimally placed. Rather than being implanted at a site near to the stimulation site, the hermetic case must instead be placed where there is suitable space and support. This in turn often requires that the lead between the electrode array and the electronics case is longer than might otherwise be required, and the lead may then be more difficult to implant.

An example of the hermetic case being distal form the stimulation site may be seen in the case of a Deep Brain Stimulator (DBS). The stimulation site is in the brain itself, but the electronics package with a connector is usually around 15 cubic centimetres in volume. Such a device cannot be implanted on the head, and so it is typically implanted on the pectoral muscle instead. A connecting lead, which may be more than 40 cm long, is implanted between the head mounted electrode array and the chest mounted electronics package by a tunnelling method. Apart from the possibility of complications if the lead is implanted too tightly, this arrangement is more complex and time consuming to implant than is desirable.

Electrical stimulation of the spinal cord induces pain relieving paraesthesia in patients with various forms of chronic pain. A number of fully implantable stimulation systems are commercially available. A schematic of a typical SCS system is shown in FIG. 1. The SCS system consists of an IPG (Implantable pulse generator) 3 and electrode(s) 8 coupled to the IPG and designed to be inserted into the epidural space of the spinal cord. There exist a large number of SCS electrode types which have been described and these generally fall into two classes, percutaneous and paddle electrodes. SCS systems employ a fixed number of electrodes, typically numbering in the range of 2 to 20 electrodes. Each electrode is contacted by a wire and terminated at corresponding feed-through connection 4 on an implant. The feed-through 4 and header connector assembly 6 form a significant volume of the device and a connection point is required for each electrode in the system.

The system is programmed via an external device 1, which may include an inductive transmitter 21 designed to provide power to an inductive receiver in order to charge an implanted rechargeable power source 24. A second RF link formed by communication between an external transceiver 11 and internal transceiver 13 is used to send data back and forth from the external control unit to the implant. The data is used to set parameters within the device and receive data from the device (for instance impedance telemetry).

The mechanical arrangement of the implantable pulse generator (IPG) of the system of FIG. 1 is depicted in FIG. 2. FIG. 2a is an end view of the body 30 and header 32 of the IPG. FIG. 2b is a side view of the IPG and associated electrode array. The stimulator body 30 is usually made from titanium, and a header 32 is connected to the implant housing. The header contains a number of contacts 36 which are electrically connected to a multichannel feed-through 4. For each channel (electrode 35) in the stimulating device there is one electrical connection to the feed-through 4 and one electrical contact 36. The electrical contacts 36 are designed to connect the signals generated inside the IPG to the electrodes 35 along the electrode array 33. The array consists of a number of stimulating sites 35 arranged in a pattern along the array to produce the desired electric field in the target tissue. The electrode array is terminated by a connector region which consists of connector elements 37 so arranged as to mate with the corresponding connector rings 36 on the implant housing.

Such a SCS device when implanted is designed to deliver a therapeutic electrical stimulus to the neural tissue. The electrical stimulus is adjusted to produce an effect and in most cases this generates an action potential in target neurons. The target nerve cells have a variety of shapes and sizes and as a result have different sensitivities to the electric field applied to the nerve. The cells have the property that at rest the membrane potential is slightly negative (˜70 mV). When the potential is shifted positively (to around 50 mV), for instance by the application of an electric field, this is known as membrane depolarisation. Sodium channels open when the potential reaches the excitation potential of the cell and the movement of Na cations into the cell causes the membrane potential to swing positive (up to 100 mV). The Na channels then close and the resting membrane potential returns by leakage of Na ions through the membrane. The change in potentials from rest through to positive and then slow relaxation back to rest is referred to as an action potential. The period from Na channel opening to recovery is known as the refractory period, during which time the neuron cannot produce another action potential.

The spinal cord is the main conduit of neural circuitry from the brain to all the organs and extremities of the body. The target of electrical stimulation of the spinal cord for the treatment of pain is sensory nerve fibres which carry pain signals from the extremities up the spinal cord to the brain. Referring to FIG. 3, there are both large diameter afferent nerve fibres 38 and small diameter afferent nerve fibres 39 which carry sensory information. Small fibres 39 carry pain and information about temperature and the large fibres 38 carry other sensory information such as touch, joint position and vibration.

The small diameter and large diameter fibres enter the spinal cord at the dorsal root but only the large diameter afferent fibres contribute branches to the dorsal columns. The “gate control theory of pain” (R. Melzack, P. D. Wall) asserts that activation of nerves which do not transmit pain signals, called nonnnociceptive fibres, can interfere with signals from pain fibres, thereby inhibiting pain.

The afferent pain-receptive nerves comprise at least two kinds of fibres: a fast, relatively thick, myelinated “Aδ” fibre that carries messages quickly with intense pain, and a small, unmyelinated, slow “C” fibre that carries the longer-term throbbing and chronic pain (both labelled 39 in FIG. 3.) Large-diameter Aβ fibres 38 are nonnociceptive (do not transmit pain stimuli) and inhibit the effects of firing by Aδ and C fibres.

The peripheral nervous system has centres in the dorsal horn of the spinal cord that are involved in receiving pain stimuli from Aδ and C fibres, called laminae. They also receive input from Aβ fibres 38. The nonnociceptive fibres indirectly inhibit the effects of the pain fibres, ‘closing a gate’ to the transmission of their stimuli. In other parts of the laminae, pain fibres also inhibit the effects of nonnociceptive fibres, ‘opening the gate’.

An inhibitory connection may exist with Aβ and C fibres, which may form a synapse 20 on the same projection neuron 40. The same neurons may also form synapses with an inhibitory interneuron 41 that also synapses on the projection neuron, reducing the chance that the latter will fire and transmit pain stimuli to the brain. The inhibitory interneuron fires spontaneously. The C fibre's synapse would inhibit the inhibitory interneuron, indirectly increasing the projection neuron's chance of firing. The Aβ fibre, on the other hand, forms an excitatory connection with the inhibitory interneuron, thus decreasing the projection neuron's chance of firing (like the C fibre, the Aβ fibre also has an excitatory connection on the projection neuron itself). Thus, depending on the relative rates of firing of C and Aβ fibres, the firing of the nonnociceptive fibre may inhibit the firing of the projection neuron and the transmission of pain stimuli.

Gate control theory thus offers an explanation of how a stimulus that activates only nonnociceptive nerves can inhibit pain. The pain seems to be lessened when the area is rubbed because activation of nonnociceptive fibres inhibits the firing of nociceptive ones in the laminae. In transcutaneous electrical stimulation (TENS), nonnociceptive fibres are selectively stimulated with electrodes in order to produce this effect and thereby lessen pain.

The precise mechanism of action of spinal cord stimulation is still the subject of study and debate. The current view is that the effect of SCS is mediated by a complex set of interactions which occur at several levels of the nervous system. SCS appears to restore normal levels of GABA in the dorsal horn, however the gate control theory of pain still appears to be the underlying mechanism of transmission.

As shown in FIG. 3b, the spine is divided into the Cervical (C) 42, thoracic (T) 43, lumbar (L) 44 and Sacral (S) 45 regions. The spinal nerve roots that enter the spinal cord at the different levels correspond to dermatomes of the body. As a result of this organisation and gate control, stimulation of the large diameter fibres can prevent transmission of pain via the small diameter fibres from specific regions of the body, so that a specific region can be selected by appropriate placement of the stimulating electrode.

Several types of stimulators and electrode systems are available. Paddle electrodes are placed in the epidural space across the dura and present a number of rows of stimulation sites to the spinal cord. These devices are implanted via a laminectomy procedure.

There are a large number of potential uses and indications for spinal cord stimulation which include but are not limited to chronic leg pain and failed back surgery, and more recently in Parkinson's disease where spinal cord stimulation has been shown to restore locomotion in animals with the condition.

The normal procedure for spinal cord stimulation is to perform an assessment phase with a trial stimulator, to assess whether the candidate receives appropriate pain relief from using the system. The clinicians must determine the stimulation level and the location of stimulation to provide effective paraesthesia to the area desired. The stimulation may be either voltage controlled or current controlled. The stimulus parameters are voltage or current level, pulse width, and frequency. The current flows from one electrode to an adjacent electrode or several adjacent electrodes on the implanted array.

One stimulation method which has been defined is known as transverse tripolar stimulation, and involves the current flowing from a central electrode to near adjacent electrodes to sharpen or focus the electric field on a desired area. Theoretical and clinical findings correlate and produce favourable thresholds and results.

Voltage controlled stimulation has the disadvantage that the amount of stimulation and corresponding level of paraesthesia can change over time. This effect is due to changes in electrode impedance which occur over time and most profoundly shortly after implantation due to the fibrous tissue encapsulation of the electrode array. Constant current stimulation avoids this issue by using a current source, whereby voltage is adjusted to supply a constant current and as a result the system is insensitive to changes in electrode impedance. The disadvantage of constant current devices is the power consumption is higher.

U.S. Pat. No 4,628,934 (Pohndorf) teaches an electronic electrode switching/selection circuit which minimizes the number of feed-throughs from a pacemaker case to a pacemaker electrode. This patent describes the selection of an electrode to be connected to one of the pins forming a feed-through into the pacemaker, and in this manner only two pins are required to interface to a number of electrode pads.

US 2008/0021292 (Stypulkowski) contemplates a system with a pulse generator connected to an extension unit that multiplexes the pulse generator between multiple electrodes. A three-wire connection joins the pulse generator to the extension unit. Stypulkowski places the pulse generator separate from the electrode array. In Stypulkowski's design, the extension electrically connects the output sources to a portion of the electrodes. The output source is contained in the implantable pulse generator. The tripolar stimulation method as described by Holshiemer could not be implemented with this scheme.

In typical SCS systems, and in the Pohndorf and Stypulkowski systems, the stimulus generation is performed by circuitry in the IPG.

Turning now to deep brain stimulation (DBS), it is noted that DBS has been used for the treatment of a range of disorders. However there are a number of complications and hazards associated with the use of these devices, such as hardware-related complications involving electrodes, lead fractures, lead migrations, short or open circuits, erosions and/or infections, foreign body reactions, and cerebrospinal fluid leaks. The hardware-related complication rate per electrode-year in one study was 8.4%, and the most common complications were related to the electrode connectors. Much of the complexity of the current devices is associated with the size of the implant and battery, which necessitates the placement of the device in the chest. A common location is in the infra-clavicular 1 cm below the clavicle. The electrodes are placed stereo-tactically into the brain, and leads and lead extensions are implanted under the skin and from the top of the head all the way down the neck to the position of the stimulator. The leads run parallel to the neck and are subject to the movements of the neck and head, and failure of the connector appears more frequent when the connector is located below the mastoid due to head movement.

The number of channels of stimulation which can be achieved by such a DBS system is low (usually 4 channels per lead) due to the requirement to balance the need for strength and fatigue resistance with flexibility and size of the cable. Increasing the number of channels requires an increase in the number of electrical conductors in the leads and hence an increase in the stiffness of the cable. Another significant contribution to the overall volume of the DBS controller device comes from the connectors and header on the implant housing which are used to connect the electrode array.

A technique has been recently proposed to allow transmission of data and power across two wires in an implanted system, described in International patent application No. PCT/US2010/042456, published on 27 Jan. 2011 as WO2011/011327 (“Single”), the content of which is incorporated herein by reference. In Single, each electrode in a multi electrode array is permanently connected to an electrode cell, which in turn is connected to a two wire bus via an implantable connector.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

According to a first aspect the present invention provides a distributed implantable neurostimulation system, the system comprising:
at least one electrode array, each array comprising at least one electrode configured to be positioned at a desired implant location within the body;
an implantable control unit configured to selectively direct stimulus and/or telemetry instructions and power to each electrode of each array;
a shared bus extending to each of the plurality of electrode arrays, the bus interconnecting each array with the implantable control unit; and
at least one electrode cell associated with each electrode array, the electrode cell obtaining electrical power and command signals from the shared bus, and controlling operation of the or each electrode associated with that electrode cell,
wherein the bus is connected to at least one of the control unit and electrode cell by docking contacts of the bus to form electrical contact with contacts of the at least one of the control unit and electrode cell.

According to a second aspect the present invention provides an implantable control unit for a distributed implantable neurostimulation system, the control unit comprising:
control circuitry configured to selectively direct stimulus and/or telemetry instructions and power via a shared bus to each electrode of each array of a distributed implantable neurostimulation system; and
a header block presenting contacts against which contacts of a bus may be docked to form electrical contact between the control circuitry and the bus, the contacts extending from the circuitry through a feed-through to the header block.

According to a third aspect the present invention provides an electrode controller for a distributed implantable neurostimulation system, the electrode controller comprising:
control logic configured to obtain power and command signals from a shared bus;
bus-interface contacts against which contacts of a bus may be docked to form electrical contact between the control logic and the bus; and
electrode-interface connections for passing electrical stimuli to respective electrodes under control of the control logic.

According to a fourth aspect the present invention provides a method of constructing a distributed implantable neurostimulation system, the method comprising:
docking contacts of a bus to form electrical contact between an implantable control unit and at least one electrode cell, the electrode cell controlling at least one associated electrode for delivering neural stimuli.

In some embodiments, the contacts of the control unit and/or electrode cell may comprise two contacts to connect a two-wire bus. The contacts of the control unit and/or electrode cell may be configured for a plug-and-socket connection, wherein the contacts are each formed about a cavity for receiving an interface module of the bus, the interface module having corresponding contacts for connecting bus wires to the respective header block contacts when the interface module is plugged into the socket cavity. In such embodiments the contacts may be configured to substantially encircle the cavity so as to effect a rotation insensitive connection.

The docking connections may comprise plug-and-socket docking or any other suitable docking engagement which effects the desired electrical connections. The docking engagement is preferably resistant to physical forces experienced in the desired implant location, for example arising during physical activity of the implantee. Once docked, the docking engagement is preferably suitably sealed to prevent ingress of body tissues, avoid creating infection sites, and prevent egress of electrical currents and the like.

In some embodiments of the invention, the electrode controller may be positioned distal from the interface module which engages with the control unit, the electrode controller being connected to the interface module by a wired bus connector lead. In such embodiments, the electrode controller is preferably positioned at a fixing site proximal to the target electrode site, for example the electrode controller may be positioned to be fixed at a surgically formed entry to the epidural space or may be positioned to be fixed to the cranium at a surgically formed burr site. The bus lead between the control unit and the electrode controller in such embodiments should be a suitable length to pass bus signals from a control unit implantation target site to the electrode controller target site, while the electrode array should be a suitable length to extend from the electrode controller fixing site to the target electrode site. As the bus lead has few wires and thus can be made more pliable, and as the electrode controller may be anchored at the fixing site, such embodiments provide for reduced mechanical disruption being passed to the electrode array, reducing the risk of electrode migration.

In alternative embodiments, the electrode controller may be integral with the interface module. Such embodiments permit the header block of the control unit to have a small number of feed-throughs and contacts, easing space concerns, while permitting a potentially large number of electrodes to nevertheless be controlled by the control unit.

In preferred embodiments, all active elements of the electrode controller are fabricated upon a single circuit board, and apart from contacts and casing the electrode controller does not have any additional components such as batteries or antennas. Such embodiments permit the electrode controller device to be made sufficiently small that it can be housed entirely within the body of a connector for interconnecting portions of the bus.

In some embodiments of the invention, the electrode controller may be configured to connect to an upstream portion of the bus in order to obtain data and power from the control unit, while also being configured to connect to a downstream portion of the bus so as to allow bus signals to pass from the control unit downstream to other electrode controllers. Each such electrical connection may be effected by docking contacts. Together with simple bus branching, such embodiments enable construction of varied and potentially complex bus architectures and associated implanted systems.

The neurostimulation device may be a spinal cord stimulation device. In such embodiments the electrode controller is preferably positioned at an entry to the epidural space, such that a bus comprising few wires extends from the controller to the electrode controller externally of the epidural space, and a plurality of electrode leads extend from the electrode controller to the electrode array internally of the epidural space. Such embodiments may mitigate lead movement externally of the epidural space by providing a flexible thin bus lead externally of the epidural space.

Some embodiments of the present invention may thus provide a device architecture which improves or at least gives an alternative for spinal cord stimulation while providing for a reduced package size and therefore minimally invasive implantation procedures for devices, while also permitting significantly increased channel counts.

Notably, the present invention does not require direct connection of electrodes to respective feed-through pins of a control unit. Rather, the present invention uses a bus of reduced wire count (e.g. two wires) which carries power and data from the implant unit to the electrode controller, the latter generating the actual stimulation signal delivered by an electrode. Telemetry data may similarly be conveyed in the opposite direction.

The present invention thus recognises that it is possible to implement circuitry for recovering power and data and generating a therapeutic signal in a single integrated circuit with no off chip components, which has an appropriately small size to fabricate as an integral part of a connector assembly. Such an assembly has the advantages of reduced size and complexity when compared with multi-pin connectors. The functionality of the electrode controller can be achieved on a single (or multiple) integrated circuit, which gives high reliability. Moreover if the implant controller is exchanged for any reason, for example replacement because a battery has reached end of life, the electrode controller can remain implanted with the electrode array and remain, reducing surgical complexity.

The bus may comprise two wires, or more than two wires, with each connector having a corresponding number of contacts.

Control circuitry in the control unit and/or electrode controller may be provided by a processor or programmable array.

### BRIEF DESCRIPTION OF THE DRAWINGS

An example of the invention will now be described with reference to the accompanying drawings, in which:

FIG. 1 is a schematic of a typical spinal cord stimulation (SCS) system;

FIG. 2 depicts the mechanical arrangement of the implantable pulse generator (IPG) of the system of FIG. 1, with FIG. 2a being an end view of the body and header of the IPG, and FIG. 2b being a side view of the IPG and associated electrode array;

FIGS. 3a and 3b are transverse and sagittal views of the human spinal column, respectively;

FIG. 4 is a schematic of an implantable neurostimulation system in accordance with one embodiment of the present invention;

FIG. 5 illustrates the mechanical layout of one embodiment of the system of FIG. 4;

FIG. 6 depicts the mechanical configuration of another embodiment of the system of FIG. 4;

FIGS. 7a to 7c detail the two wire interface architecture used in the embodiments of FIGS. 5 and 6;

FIGS. 8a to 8c illustrate the mechanical configuration of two-contact components in accordance with some embodiments of the invention;

FIGS. 9a-9c depict exploded views of ceramic and MEMS hermetic implantable cases, in accordance with respective embodiments of the invention;

FIGS. 10a and 10b are exploded and perspective views, respectively, of a connector in accordance with a further embodiment of the present invention;

FIGS. 11a and 11b illustrate an implant system configuration in accordance with one embodiment of the invention;

FIGS. 12a-12e illustrate a distributed DBS implant system in accordance with another embodiment of the invention; and

FIG. 13a-13c illustrate the construction of varied system architectures using components in accordance with the present invention.

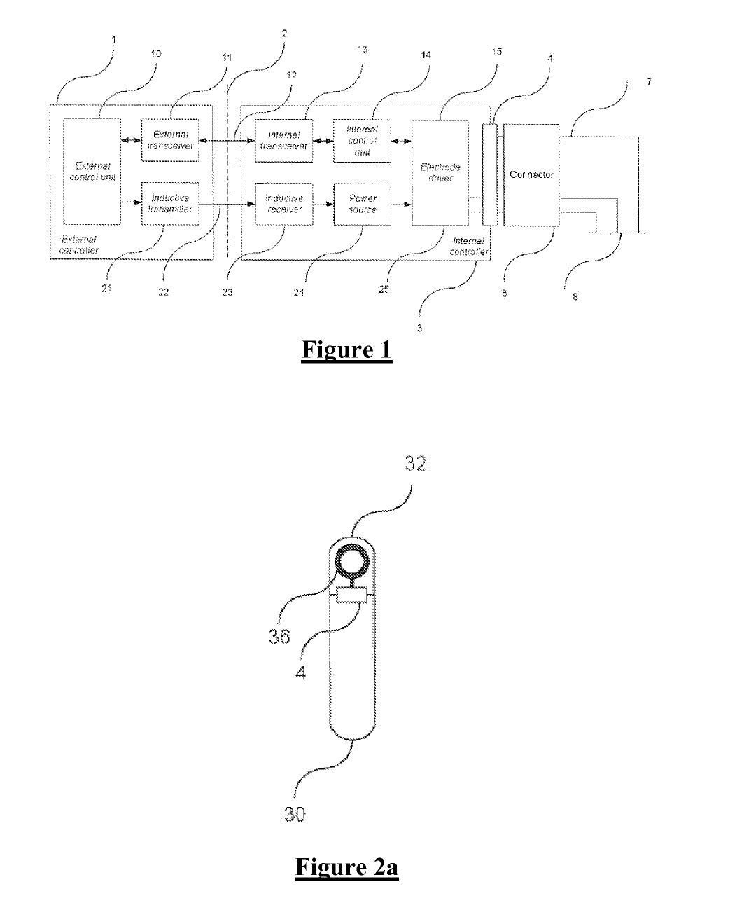

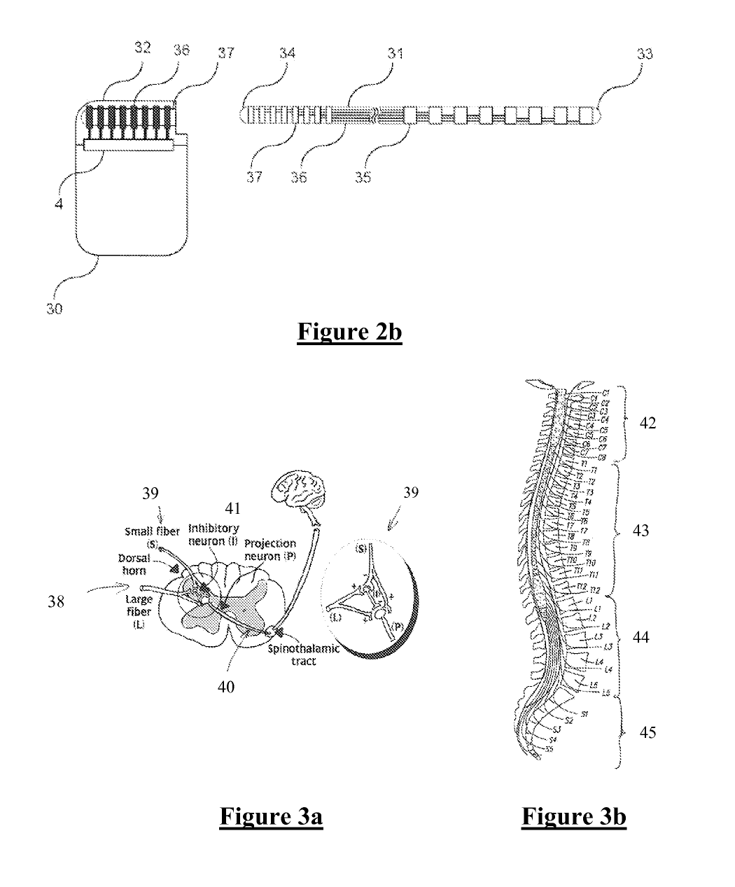

### DESCRIPTION OF THE PREFERRED EMBODIMENTS

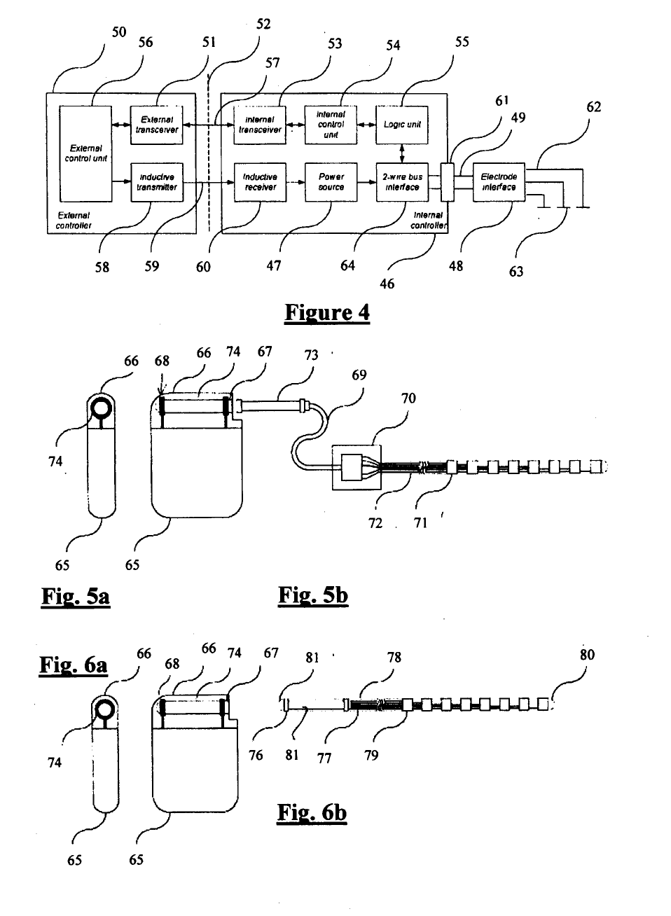

A schematic of an implantable neurostimulation system is shown in FIG. 4. A two wire bus system is connected to the implant controller (IC) 46. IC 46 contains a DC power source 47 (e.g. implantable rechargeable lithium ion battery, a thin film lithium ion battery or primary cell), or power may be inductively transmitted to the IC. An alternator generates an AC signal from the power source for supply of power to the electrode cell 48. Data is also transmitted to the electrode cell, in the manner described in Single, WO2011/011327, although it is to be noted that in alternative embodiments of the present invention an alternative bus protocol may be adopted for data and/or power transfer. The data from the IC 46 is decoded in the logic of the electrode cell 48 and this data specifies the generation of therapeutic signals, such as a charge balanced biphasic stimulus, to be delivered by the electrode(s) 63 associated with the cell 48.

In FIG. 4 external device 50 communicates bidirectionally with the implanted device. Several types of external device may interact with the implanted device. The external device may be an interface to a programming system which is used for setting patient parameters or adjusting the system. The external device 50 may be a control unit used by the recipient of the implant for the purposes of adjustment. The control unit may be used to operate the implant, switch it on or off, or may be used to adjust parameters within a specified range within the implant unit. The external unit may contain visual feedback in the form of a display screen for the user to adjust the implant's behaviour. In a further embodiment the external device may perform some processing of data received from the implant and then on the basis of the processing adjust the operation of the implant. This may be done in an automatic manner whenever the external device is in close proximity to the implanted device and a communications link can be established.

The external device communicates with the internal device using a transceiver 51. The transceiver is a radio frequency transceiver which operates in the MICS band. Signals from the external unit are received by the internal transceiver unit 53 and decoded by the control unit 54. The control unit 54 may be a microprocessor of a suitable type eg MSP 430 from Texas Instruments. The processor supervises the function of the implant including the status of the power source.

The microprocessor is interfaced to a logic unit 55. The logic unit 55 in this embodiment is an electrically programmable gate array which can be programmed by the processor which is connected to it. This has a number of advantages including the ability to change the function of the interface. The two wire bus interface 64 generates signals and commands which are communicated externally from the implant housing 46 via a two pin feed-through 61. The two pin feed-through is connected by a connector, which may for example be many centimetres in length, to electrode cell 48 which decodes the commands and creates stimulation sequences for application to an array of electrodes 63, which are positioned in the spinal cord to stimulate the tissue.

The electrode cell 48 can also generate signals to be transmitted back to the implant 46 via the two wire bus 49 for processing within the implant unit 46 and/or for transmission by internal transceiver 53 out of the body to an external device 50.

The present embodiment thus provides for a multi-channel electrode array 63 to be connected to an IC with only two connection points which in turn means only two feed-throughs (61) and a two channel connector in order to make the contact.

The mechanical layout of the device of FIG. 4 is shown diagrammatically in FIG. 5. An implant housing 65 is connected to a header block 66 which contains two connection points 67 and 68. The header block 66 is adapted to receive in socket cavity 74 a two pin connector assembly 73 which is designed to make electrical contact to contact points 67 and 68. The two contacts of assembly 73 are connected via lead 69 to a second hermetic package 70 referred to as an electrode controller or electrode cell (EC). This hermetic package contains an integrated circuit which implements the two wire bus power and data protocol. The electrode array 71 is connected to this module and conducting elements are used to transmit signals from the electrode controller 70 to each corresponding electrode.

FIG. 6 depicts the mechanical configuration of another embodiment of the invention. Reference numerals repeated from FIG. 5 refer to like components in FIG. 6, and discussion of such components is not repeated here. In the embodiment of FIG. 6, rather than having an electrode controller 70 separate to the plug 73 as occurs in the embodiment of FIG. 5, the electrode controller in the embodiment of FIG. 6 is instead contained in a small package 81. Package 81 is designed to serve as part of the plug and to mate with the header 66 directly. Each electrode 79 is wired to a corresponding contact on the package 81. This package 81 thus contains the electrode cell (EC) electronic circuit which is connected to the IC 46 via two pins in the connector.

In another embodiment, the mechanical configuration may be such that one or more electrode controllers may be packaged in a manner which allows them to be disposed inside the lead structure close to each electrode area.

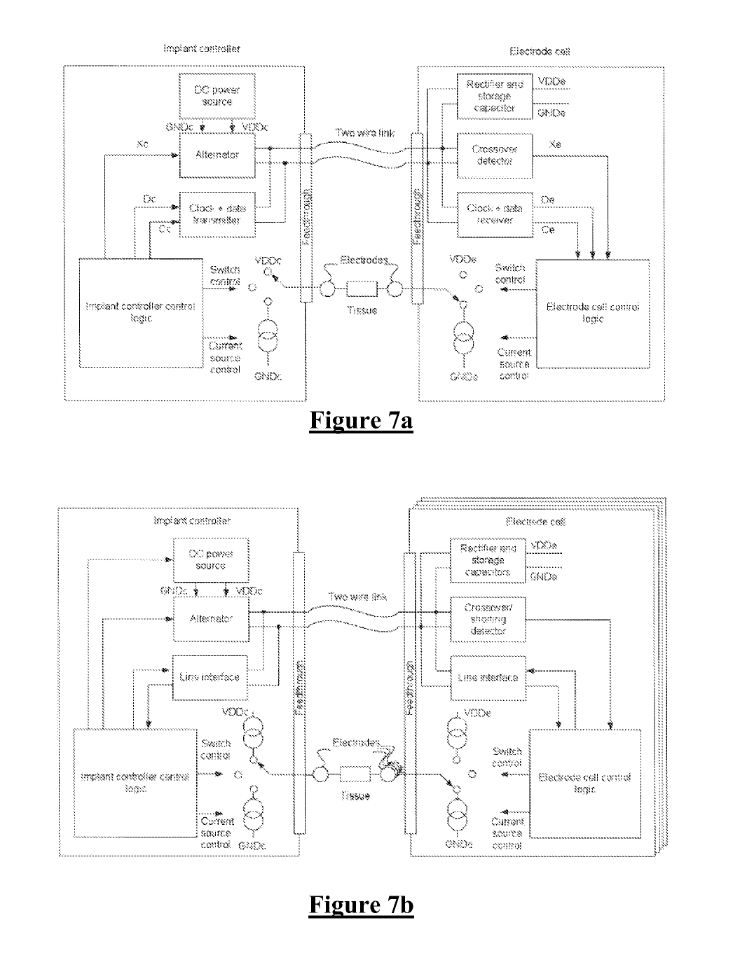

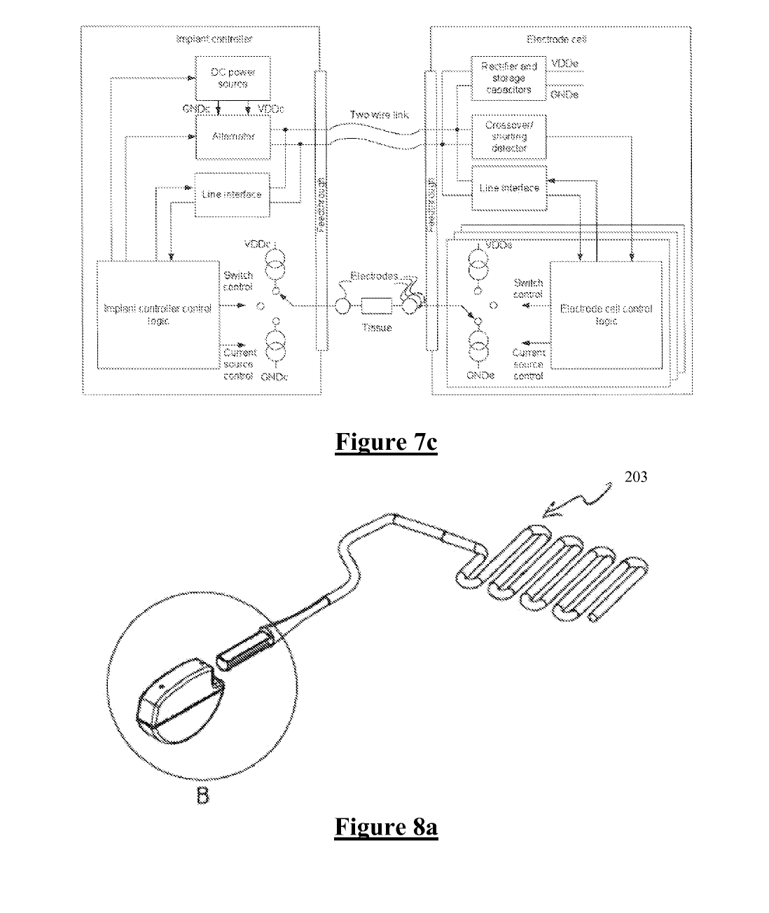

The two wire interface architecture used in these embodiments is detailed in FIGS. 7a to 7c. An alternator in the implant controller is used to generate AC voltages and a receiver rectifier in the electrode cell is used to generate power required by the cell to perform its function. The system as designed allows for many electrode cells to receive power and data. FIG. 7a illustrates an embodiment in which a single electrode cell controls a single electrode, with all power and data being transferred to the electrode cell by a single two wire link shared bus. FIG. 7b illustrates another embodiment in which a plurality of electrode cells each control a single electrode, with all power and data being transferred to the electrode cells by a single two wire link shared bus. FIG. 7c details the architecture used to implement an electrode controller (EC), with a single electrode cell controlling a plurality of electrodes, with all power and data being transferred to the electrode cell by a single two wire link shared bus. In the embodiment of FIG. 7c each electrode in a multi-channel electrode array is connected to its own current source and switch, which in turn is connected to a unique electrode cell control logic dedicated for that electrode.

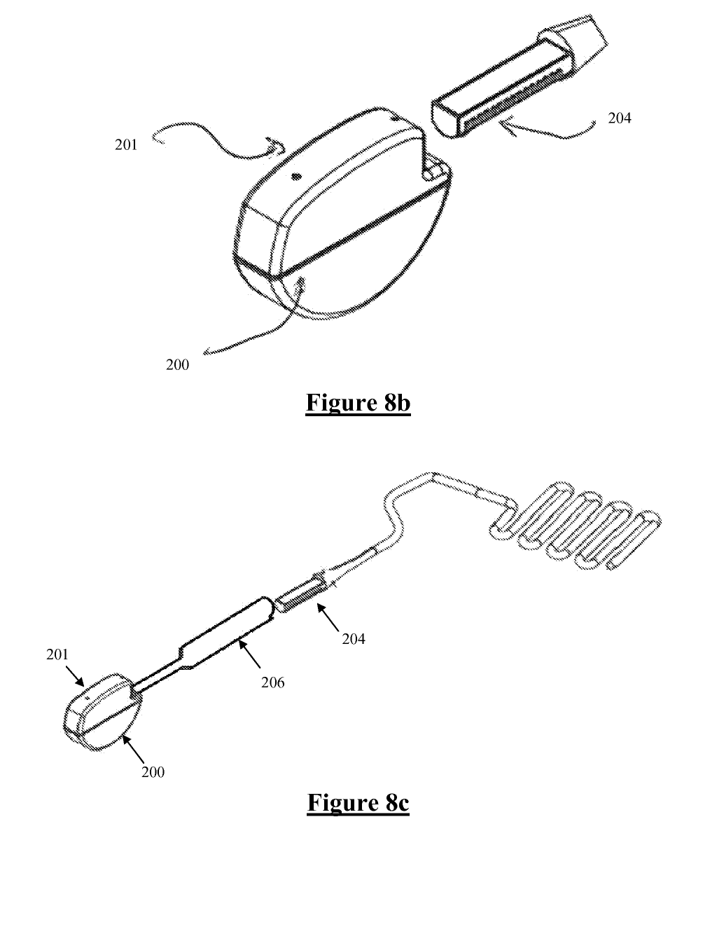

FIGS. 8a and 8b illustrate the mechanical configuration of an embodiment in which the electrode bus interface is housed in a connector unit which is designed to be interfaced to a header of an implant body, similarly as for the embodiment of FIG. 6. The hermetic implant body 200, which contains an electronics module and battery, and the header assembly 201 are separable from the electrode assembly 203. An electrical connection is made between the implant electronics and the electrode controller module 204 via two contacts. The hermetic capsule of the module 204 has two types of connection. One type of connection is hard wired to an electrode contact, with the embodiment of FIG. 8 having 32 such connections. The second type of connection forms a connector ring around the case of module 24 which enables the module 204 to form a contact through a connector to the implant 201. Thus, the module 204 in FIG. 8 has two dis-connectable connections to the header block 201 and 32 permanent connections to the electrodes of the array 203. Suitable compact fabrication of module 204 in this way permits a reduction in the overall size of the implant. The contacts of module 204 can be formed by any suitable technique, such as a BAL seal that consists of a circular spring contact. The connector design can be made to conform to the IS-1 standard ISO 5841-3-2000 or design variant or other connector methods. An alternative knitted assembly connector arrangement as described in US Patent Application Publication No. 2010/0070007 may be utilised.

FIG. 8c illustrates a further embodiment of the invention in which the controller 204 is distal from the implant body 200, and is plugged into a lead mounted socket 206, which in turn is connected to the header block 201.

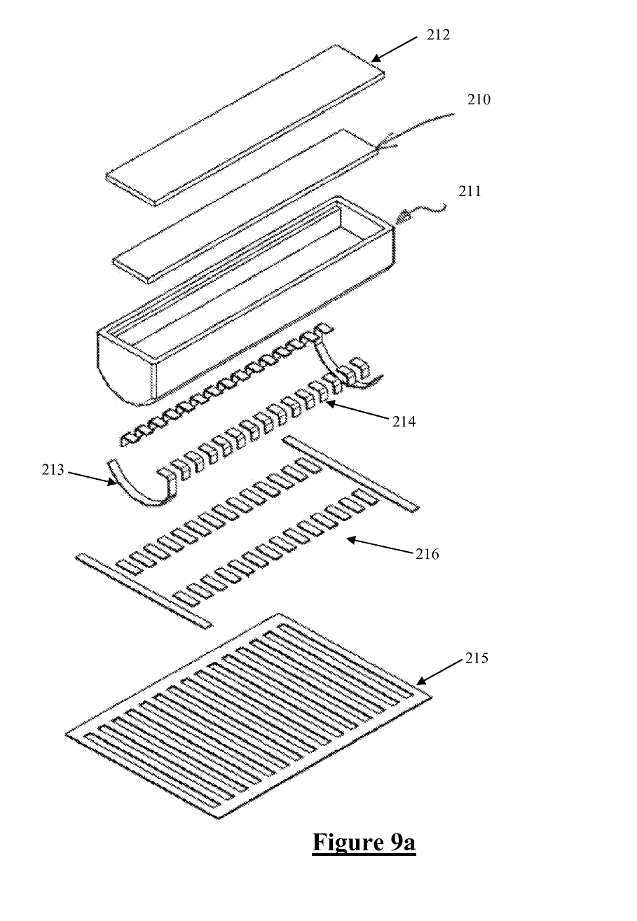

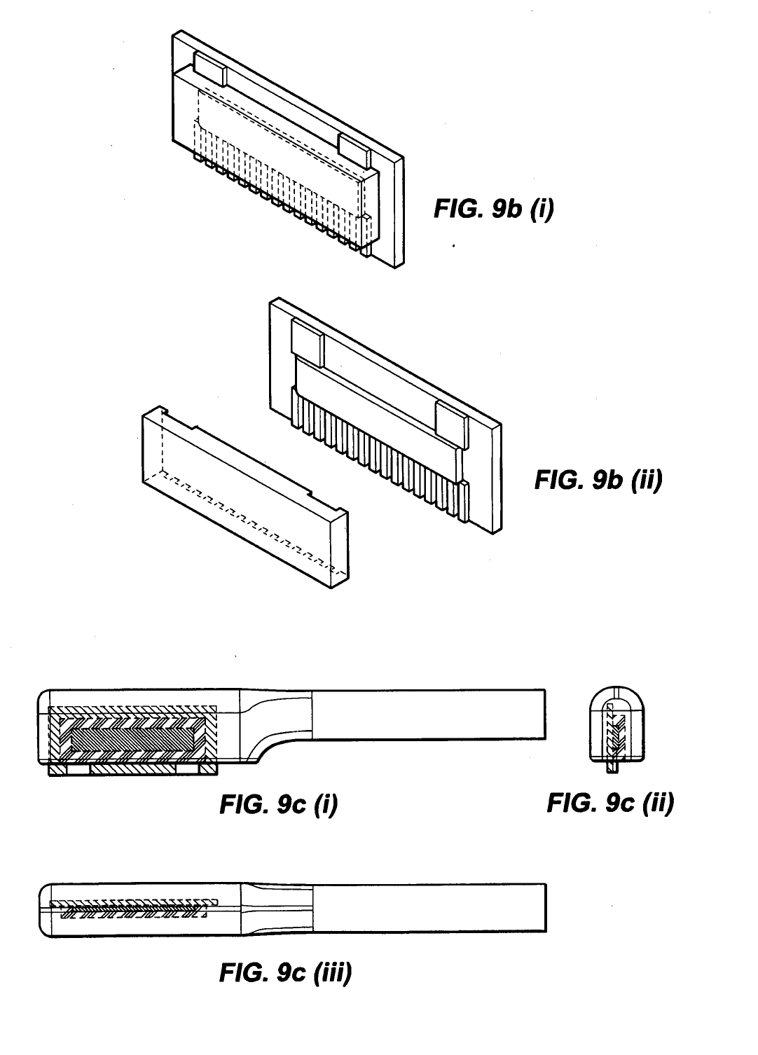

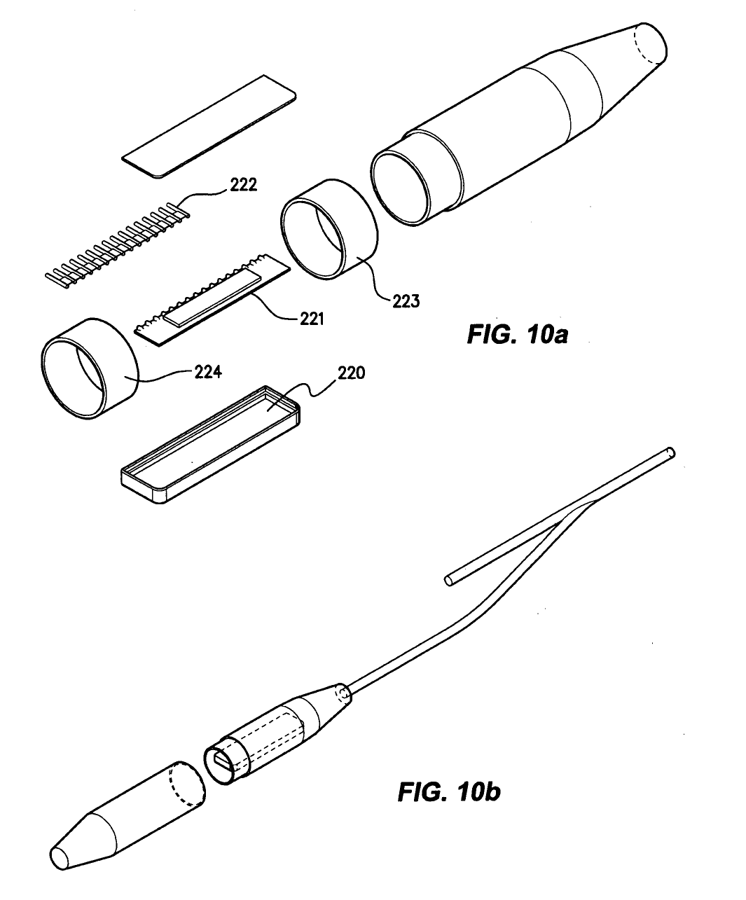

There are several options for fabrication of the hermetic implantable case which contains the (EC) electronics. In preferred embodiments the electrode cell package contains one or more integrated circuits, and no other components. This circuit could be packaged in a ceramic case as shown in FIG. 9a or a titanium case (FIG. 10a) or a MEMS case (FIG. 9b). The case depicted in FIG. 9a is preferably fabricated from ceramic via powder injection molding. FIG. 9a depicts an exploded view and shows the integrated circuit 210. The package consists of a lid 212 made from a suitable bio-compatible material which can be hermetically sealed to the case 211. Contacts are disposed along the side of the case 211 to create a connection between the internal hermetically sealed portion and the external environment. The case 211 is preferably formed by injection moulding a ceramic material around the contacts. Two types of contact are illustrated 213 and 214. Contact 213 extends around the outside of the case and forms an electrically conducting wiper which forms one side of an implantable connector. The other contact type 214 is so designed to form a permanent connection between an electrode element contact and the case. The contacts 214, 213 can be formed from a stamped foil 215 which is held in place during over-moulding with ceramic and then excess material removed to form contacts 216. In alternative embodiments the module 204 and other such components may be formed by any suitable method, for example as an injection molded micro package in accordance with the teachings of International Patent Publication No. WO2011/066478, the contents of which are incorporated herein by reference.

In a second alternative a wafer level packaging technology may be employed as shown in FIG. 9b. Such a wafer level package has been described in United States Patent Publication No. 2010/0262208, the contents of which are incorporated herein by reference.

In other embodiments the hermetic implantable case could be a MEMS case as illustrated in FIG. 9b (perspective views) and 9c (side, plan, end views).

An alternative and more traditional encapsulation technique, applicable in some embodiments of the present invention, is to use a titanium metal box. FIG. 10a is an exploded view of such a connector, showing a metal box 220 which is designed to receive a circuit board 221 or a ceramic hybrid on which an integrated circuit is mounted. A linear feed-through 222 creates the electrical path from the electrode bus chip to each of the electrodes and to the two connection points 223 and 224. FIG. 10b illustrates the constructed connector. By using a single circuit board without any additional components such as batteries or antennas, the device is sufficiently small that it can be housed entirely within the connector body and is circumferentially surrounded by the two contact rings 223, 224 which are so disposed to form a connection between the module and the implant housing. The circular metal contacts beneficially result in a connector that is insensitive to rotation along the long axis of the device.

The connector assembly in the embodiment of FIG. 10 is further improved by the addition of sealing flanges (not shown) between contacts 224 and 223. A variety of configurations for the mechanical connector are possible. For instance the end contact 224 may be of a smaller diameter than 223 to facilitate insertion in the corresponding female portion of the connector. FIG. 10 depicts a flying lead style connector which interfaces to the EC. This in-line connector can be made considerably smaller than a header block for a standard implantable system. This is desirable for example so that the connector assembly can be used as an anchor for the electrode array. Lead migration is a common problem in spinal cord surgery and there have been systematic attempts to study this and look for anchoring techniques to prevent it.

The use of a hub or EC as an anchoring point helps mitigate lead migration. As the connection between the anchor point and the stimulator housing is flexible, the force imparted on the lead end is reduced, making migration less likely. Routinely during spinal cord surgery the electrode exit is secured at the point of exit from the epidural space. The hub of the present embodiments can be so designed as to form part of the exit strain relief. This has the considerable advantage that the component of the system that contains the greatest number of wires is anchored at the point closest to the exit of the lead from the epidural space. From this point extending to the stimulator the lead only contains two wires and can be more pliable, and this section of lead experiences the greatest movement as it is embedded in soft tissue and muscle and the reduction in stiffness afforded by the two wires increases the fatigue life and reduces the potential failures.

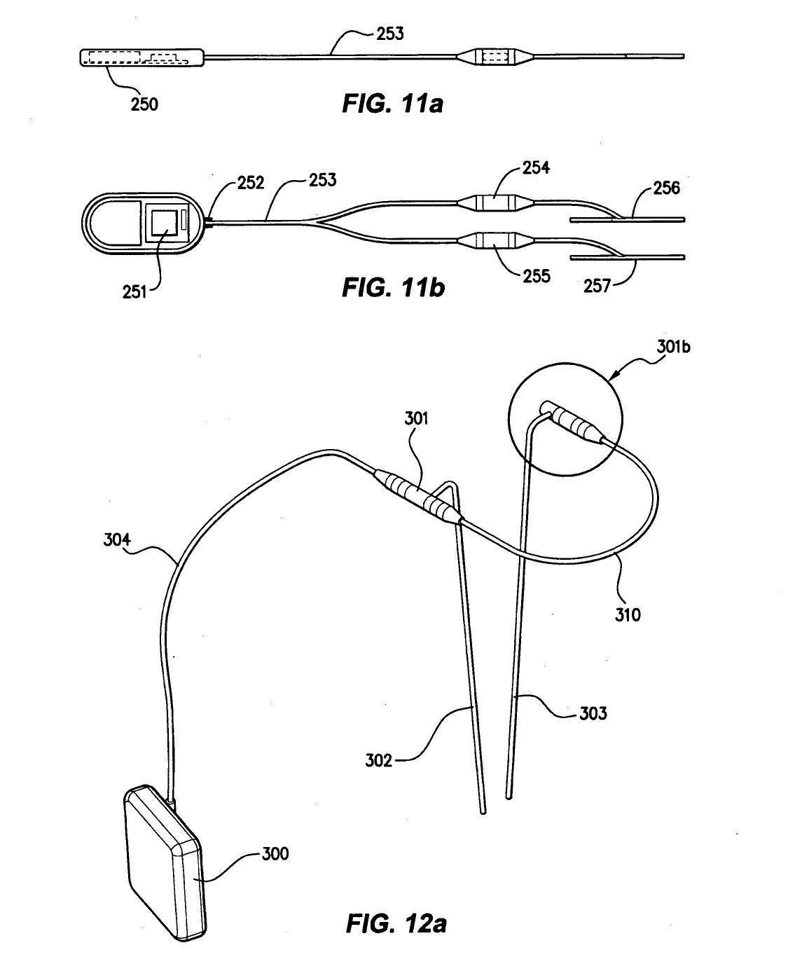

FIG. 11 illustrates an implant system configuration in accordance with one embodiment of the invention. The implant controller unit 250 contains a rechargeable battery and an electronics module 251 which implements the electrode controller as depicted in FIG. 7c. The electronics 251 and battery are contained in a conventional implant package 250 constructed from laser-welded titanium. The package 250 has two hermetic feed-throughs 252 that conduct the two wire interface signals from the electronics 251 outside the package 250 to the lead 253. The feed-throughs 252 can be fabricated by known methods such as those used by Greatbatch, Inc., of Clarence, N.Y., USA, or a feed-through could be constructed by the methods described in United States Patent Application Publication No. 2010/0258342, United States Patent Application Publication No. 2010/058126 and/or International Patent Publication No. WO2011/066477, the contents of which are incorporated herein by reference.

The lead 253 extending from the IC 251 contains two wires. As described in the preceding, a number of “electrode hubs” 254, 255 can be interfaced with a single bus 253 and as a result multiple electrode arrays 256, 257 can be added to the system by addition of extension units (FIG. 11b). The size of the electrode controllers/hubs 254, 255 is much smaller than the implant housing 250 and therefore they can be located in close proximity to the end of the respective electrode array 256, 257. The electrical connection 253 between the electrode controller 254, 255 and the implant 250 consists of two wires, and this allows the lead to be designed and constructed in such a way that it is intrinsically more robust than if it carried for its entire length the number of wires required for each electrode channel.

In accordance with the present invention, the hubs 254, 255, which convert a standard two wire bus interface to a multi electrode output, can be used to construct much more complex systems. For example, some embodiments of the invention may provide a device having a single electrode cell per electrode channel. FIG. 7b illustrates the architecture for such a device, with multiple electrode cells and one electrode per electrode cell. Each device and electrode is uniquely addressed in accordance with the teachings of WO2011/011327.

Other embodiments of the invention may be applied to effect deep brain stimulation (DBS) or early chronic cerebellar stimulation (CCS) for the treatment of pain and movement disorders. For example, some embodiments of the invention may be employed to effect one or more of: DBS for Parkinson's treatment; DBS of the internal pallidum or subthalamic nucleus to treat upper limb akinesia in Parkinson's disease; DBS for treatment of medication-refractory idiopathic generalized dystonia, DBS in treatment of Spasticity and Seizures; bilateral DBS of the internal pallidum and the subthalamic nucleus to improve motor function, movement time, and force production; DBS for the treatment of pain such as failed back syndrome, peripheral neuropathy, radiculopathy, thalamic pain, trigeminal neuropathy, traumatic spinal cord lesions, causalgic pain, phantom limb pain, and carcinoma pain; and DBS for treatment of essential tremor, for example.

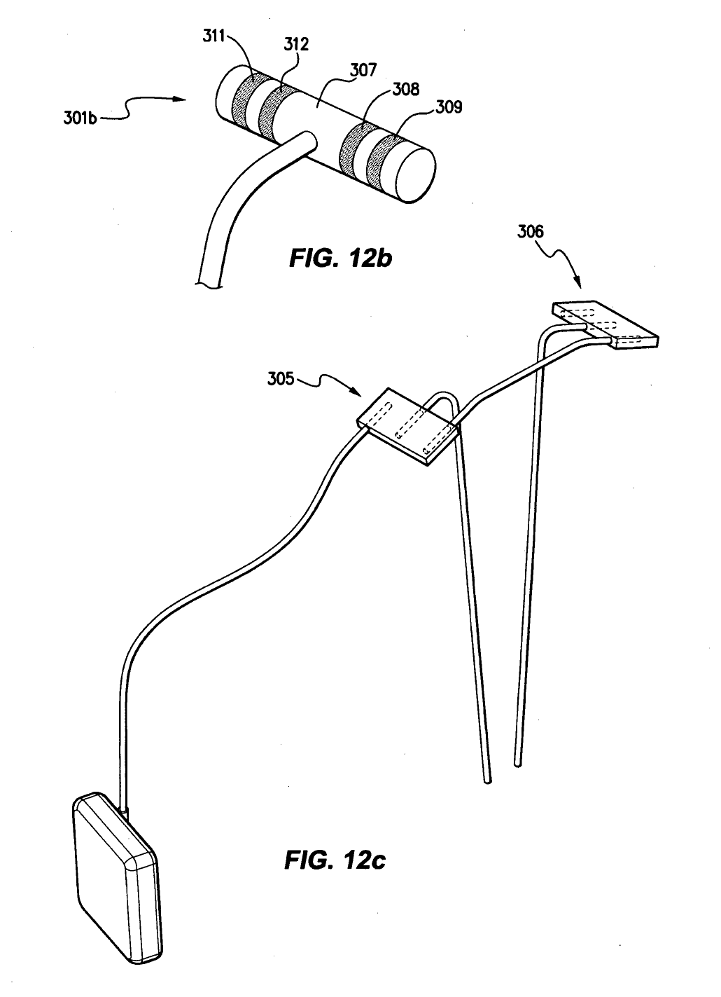

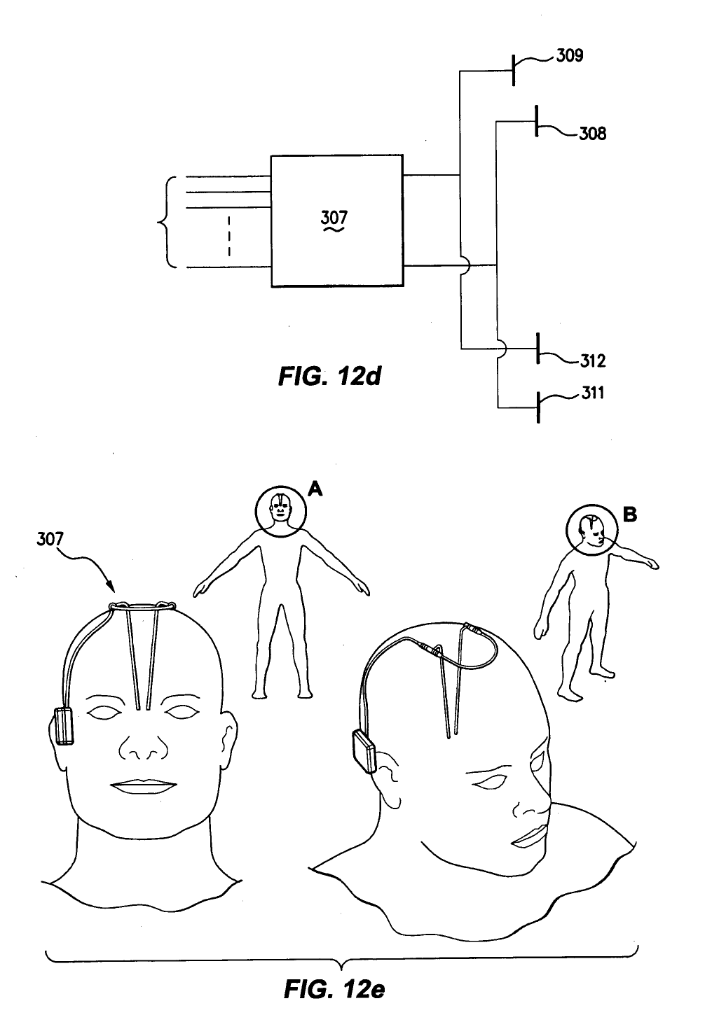

The volume of the connectors and header of conventional DBS devices can be significantly reduced in the embodiment of the present invention shown in FIG. 12a, employing a distributed architecture. In this embodiment, the implant controller 300 is designed to be located on the skull on the temporal bone site routinely used in cochlear implant implantation. Notably, the implant controller battery assembly (within case 300) is separated from the implant hubs 301, 301b via the two wire interface 304 in a similar manner to that described for the embodiments of FIGS. 5, 6 and 8-11. In more detail, the implant controller 300 is connected to a two pin connector of hub 301 with two wires in the connection 304. The DBS electrode 302 is connected to the two pin connector via the electrode hub 301. The system is readily adapted to drive a second electrode by providing an extension lead 310 from the first hub 301 to the second hub 301b, to drive electrode 303.

As shown in FIG. 12b, the electrode assemblies are terminated in this design in a “Tee” shape 307. Two pairs of annular contacts on the “Tee” (one pair being denoted at 308, 309, and the other pair denoted at 311, 312) are configured to mate either with a lead from the stimulator or to a connector 310 which can bridge from one hub 310 to another hub. The contact pads on the “Tee” connector are wired as shown in FIG. 12d, with the contacts 308, 309 being electrically connected to the electronics hub 307 and in parallel with the second pair of contacts 311, 312. This arrangement simply effects a two wire bus tap point.

The number of electrodes in the system of FIG. 12a can be extended beyond two by simply adding an additional bridging piece 310 to the unused portion of the “Tee” connector 301b, and attaching an additional electrode. The chain thus can be simply configured to consist of one or any other number of electrodes, and can be terminated by insulating the end of the terminating “Tee” piece with a suitable cap to isolate the final unused contacts from the body tissue.

The “Tee” connector of FIG. 12 in alternative embodiments of the invention may be round or any other shape suitable for the intended location on the skull. The connector 301 may have the simple geometry shown in FIG. 12a of having two connectors attached to the end of a single stimulation lead, alternatively the connector may be provided with third or additional pairs of contacts, for example to effect bus branching.

Moreover, in some embodiments the “Tee” piece and the associated electronics can be adapted for location in the burr hole formed in the skull during implantation, in order to fix the device and to secure the electrode lead wire accurately. Such configuration of the tee piece allows it to be anchored at the target location, thus preventing movement post insertion. Such embodiments may be advantageous in reducing the risk of early displacement when the electrode is disengaged from the insertion tool, or the risk of displacement of the electrode tip from its insertion position over a period of time, such as may be caused by the dynamic pulsatile nature of the brain. Some embodiments of this invention may thus improve the long term reliability of DBS devices.

Moreover, by providing the two wire buses 304, 310 between the implant controller 300 and each electrode controller 301, intra-operative repositioning of an electrode may be eased due to the more pliable nature of such two-wire leads as compared to the stiffer nature of multi-wire leads.

The systems of FIGS. 11 and 12, in providing for distributed implant systems to be built up by repeated use of a small number of component types, permits each type of component to be fabricated in a commoditised or mass production manner, reducing overall fabrication costs of potentially complex distributed systems as compared to bespoke construction of such systems.

The “Tee” connector may in alternative embodiments of the invention have an alternative geometry of orientation of the connection points, not limited to 90 degrees. For example, FIG. 12c shows an alternative configuration of tee connectors 305, 306.

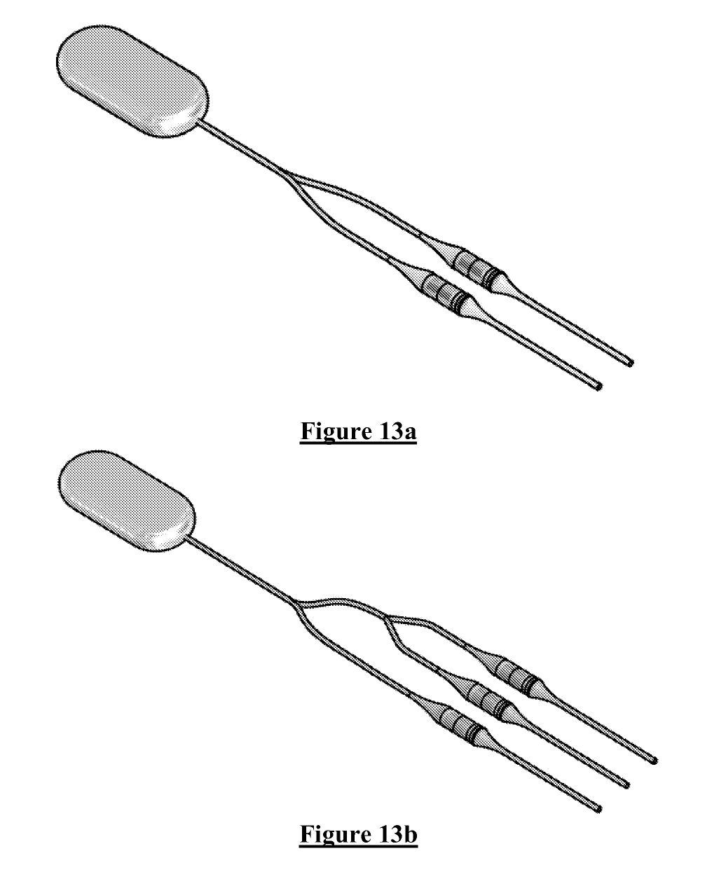

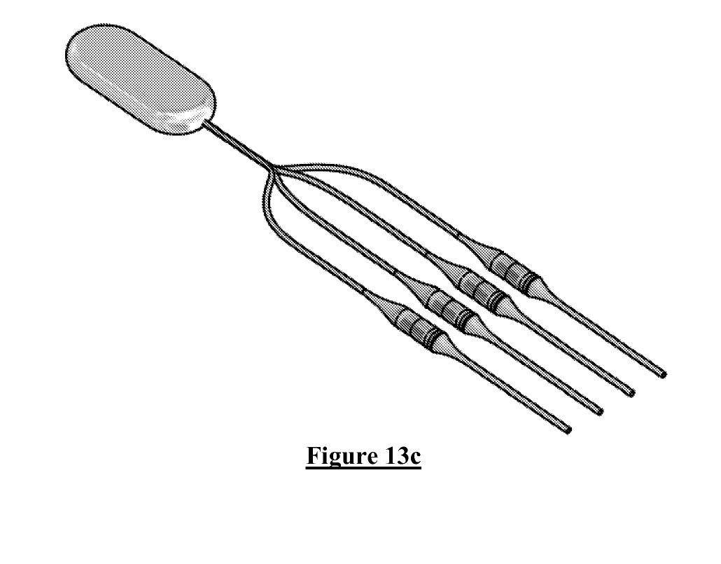

Both the spinal cord stimulation architectures of FIGS. 5-11 and the deep brain stimulation systems of FIG. 12 utilise the two wire bus architecture to allow the interface of electrode array elements to an implant controller. The number of electrode elements is not limited to two but may be many. FIG. 13a illustrates a system which consists of two electrode arrays which may each consist of 16 stimulating channels. The addition of a 2^{nd} pod and another branch allows connection of an additional electrode array to form the three-array system of FIG. 13b. Electrodes /arrays can be added to the system by adding branches or by splitting from a single junction as is illustrated in FIG. 13c. The components of the present embodiments thus permit a large range of choice in the combination of branches and splits to achieve a desired arrangement of electrodes. This flexibility in final configuration is advantageous for applications in the periphery of the human body. Such applications include stimulation of the occipital nerve for migraine, and many other potential applications.

Thus, some embodiments of the invention recognise that there are a range of potential applications of neuromodulation and neuro-stimulation devices, including the management of pain (by spinal cord stimulation, SCS), epilepsy (by vagal nerve stimulation, VNS), Parkinson's disease (by deep brain stimulation, DBS), essential tremor (by DBS), dystonia (by DBS), depression (by DBS) and cochlear implants for the treatment of profound hearing loss by auditory nerve stimulation. Moreover, such devices may in future be adapted for the treatment of a range of other disorders including neuropathic pain (through DBS and cortical stimulation, CS), epilepsy (via DBS and/or CS), and a number of different forms of head ache including occipital neuralgia migraine and cluster headaches. Psychiatric illness may also be treated with neuro-modulation and trials are under way for obsessive compulsive disorder, depression, addiction, and Tourette's syndrome. Physical disorders such as stroke, tinnitus, minimally conscious state, and hypertension are also being researched in relationship to the development of neuromodulation techniques.

Some embodiments of the invention further recognise that sensors are being developed for a variety of applications including the monitoring of intracranial pressure due to hydrocephalus and various other pressure, temperature and physiological monitoring applications.

Some embodiments of the invention may thus provide methods and means to provide the stimulating source at a location extremely close to the stimulating site, so that both the mechanical and electrical problems associated with long leads are mitigated. Embodiments of the invention may further provide for multiple electrodes to be connected, powered and addressed with only two wires, or at least with a smaller number of wires than the number of electrodes. In some embodiments, systems can be constructed with multiple packages with each package carrying a specific function and placed at a position which is more optimal for its use. Connector assemblies can be constructed with two, or a few, conductors which requires correspondingly fewer feed-throughs to the system component which is responsible for powering the system. Moreover, systems of some embodiments may be considerably smaller than can be achieved with conventional technology.

The benefits and applications of these embodiments are described for devices for spinal cord stimulation, deep brain stimulation and cochlear implants, however the present invention is not limited to such applications.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A distributed implantable neurostimulation system, the system comprising:
at least one electrode array, each array comprising at least one electrode configured to be positioned at a desired implant location within the body;
an implantable control unit configured to selectively direct stimulus and/or telemetry instructions and power to each electrode of each array;
a shared bus extending to each of the plurality of electrode arrays, the bus interconnecting each array with the implantable control unit; and
at least one electrode cell associated with each electrode array, the electrode cell obtaining electrical power and command signals from the shared bus, and controlling operation of the or each electrode associated with that electrode cell,
wherein the bus is connected to at least one of the control unit and electrode cell by docking contacts of the bus to form electrical contact with contacts of the at least one of the control unit and electrode cell.

2. The system of claim 1, wherein the electrode controller is positioned distal from the control unit.

3. The system of claim 2 wherein the electrode controller is positioned at a fixing site proximal to the target electrode site.

4. The system of claim 3 wherein the fixing site is a surgically formed entry to the epidural space.

5. The system of claim 3 wherein the fixing site is a surgically formed cranial burr hole.

6. The system of claim 1, wherein the electrode controller is positioned within an interface module configured to dock with the control unit.

7. An implantable control unit for a distributed implantable neurostimulation system, the control unit comprising:
control circuitry configured to selectively direct stimulus and/or telemetry instructions and power via a shared bus to each electrode of each array of a distributed implantable neurostimulation system; and
a header block presenting contacts against which contacts of a bus may be docked to form electrical contact between the control circuitry and the bus, the contacts extending from the circuitry through a feed-through to the header block.

8. The control unit of claim 7, wherein the contacts are each formed about a cavity for receiving an interface module of the bus in a plug-and-socket arrangement.

9. The control unit of claim 8 wherein the contacts substantially encircle the cavity so as to effect a rotation insensitive connection.

10. An electrode controller for a distributed implantable neurostimulation system, the electrode controller comprising:
control logic configured to obtain power and command signals from a shared bus;
bus-interface contacts against which contacts of a bus may be docked to form electrical contact between the control logic and the bus; and
electrode-interface connections for passing electrical stimuli to respective electrodes under control of the control logic.

11. The electrode controller of claim 10 wherein the contacts are each formed about a cavity for receiving an interface module of the bus.

12. The electrode controller of claim 11 wherein the contacts substantially encircle the cavity so as to effect a rotation insensitive connection.

13. The electrode controller of claim 10 wherein all active elements of the electrode controller are fabricated upon a single circuit board.

14. The electrode controller of claim 10 wherein the electrode controller is housed entirely within the body of a connector for interconnecting portions of an implanted bus.

15. The electrode controller of claim 10 further configured to connect to an upstream portion of the bus in order to obtain data and power from the control unit, while also being configured to connect to a downstream portion of the bus so as to allow bus signals to pass from the control unit downstream to other electrode controllers.

16. A method of constructing a distributed implantable neurostimulation system, the method comprising:
docking contacts of a bus to form electrical contact between an implantable control unit and at least one electrode cell, the electrode cell controlling at least one associated electrode for delivering neural stimuli.
